(19) United States
(12) Patent Application Publication
(10) Pub. No.: US 2011/0251492 A1
(43) Pub. Date: Oct. 13, 2011

(54) ULTRASOUND ASSESSMENT OF LUMENS TO FACILITATE REPAIR OR REPLACEMENT

(76) Inventors: David C. Forster, Los Altos Hills, CA (US); Yenyu Chen, Palo Alto, CA (US)

(21) Appl. No.: 12/904,926

(22) Filed: Oct. 14, 2010

Related U.S. Application Data
(63) Continuation-in-part of application No. 11/420,189, filed on May 24, 2006.

(51) Int. Cl. A61B 8/14

(52) U.S. Cl. 600/470; 600/466

(57) ABSTRACT

Devices and methods for assessing the orientation and shape of vessel lumens and hollow portions of organs are described. The devices and methods are particularly adapted for determining the orientation and shape of the native heart valves to facilitate the later implantation of a prosthetic heart valve. The devices are typically catheter-based having an expandable member fixed to a distal end of the catheter. Located within the expandable member is an ultrasonic imaging probe and an acoustic reflective material. The methods typically comprise deploying the balloon percutaneously to a target location, expanding the balloon, and determining the orientation and shape of a lumen, particularly a cardiac valve.

### CROSS REFERENCES TO RELATED APPLICATIONS

This application is a continuation-in-part application of U.S. patent application Ser. No. 11/420,189, entitled “Assessment of Aortic Heart Valve to Facilitate Repair or Replacement,” filed May 24, 2006, which application is hereby incorporated by reference in its entirety.

### FIELD OF THE INVENTION

The present invention relates generally to medical devices and methods. More particularly, the present invention relates to methods and devices for assessing the orientation, shape, size, topography, compliance, and other aspects of lumens and surrounding tissue. The devices and methods are particularly adapted for use during minimally invasive surgical interventions, but may also find application during surgical replacement on a stopped heart, less invasive surgical procedures on a beating heart, and other percutaneous procedures.

### BACKGROUND OF THE INVENTION

Minimally invasive surgery provides several advantages over conventional surgical procedures, including reduced recovery time, reduced surgically-induced trauma, and reduced post-surgical pain. Moreover, the expertise of surgeons performing minimally invasive surgery has increased significantly since the introduction of such techniques in the 1980s. As a result, substantial focus has been paid over the past twenty years to devices and methods for facilitating and improving minimally invasive surgical procedures.

One area in which there remains a need for substantial improvement is pre-surgical assessment of treatment locations intended to be subjected to a minimally invasive surgical procedure. For example, when a surgical procedure is to be performed at a treatment location within the body of a patient, it would frequently be beneficial for the surgeon to have prior knowledge of the shape, size, topography, compliance, and other physical properties of the treatment location. This information would be particularly useful in relation to minimally invasive surgical procedures in which prosthetic devices are implanted within a body lumen or within a hollow portion of an organ located within the body of the patient. Such information could then be used to select the size and/or shape of the prosthetic device to more closely match the size, shape, and topography of the treatment location.

A particular portion of the anatomy for which complete and accurate physical assessment would be beneficial are the coronary valves. Diseases and other disorders of heart valves affect the proper flow of blood from the heart. Two categories of heart valve disease are stenosis and incompetence. Stenosis refers to a failure of the valve to open fully, due to stiffened valve tissue. Incompetence refers to valves that cause inefficient blood circulation, permitting backflow of blood in the heart.

Medication may be used to treat some heart valve disorders, but many cases require replacement of the native valve with a prosthetic heart valve. In such cases, a thorough assessment of the shape, size, topography, compliance, and other physical properties of the native valve annulus would be extremely beneficial. Prosthetic heart valves can be used to replace any of the native heart valves (aortic, mitral, tricuspid or pulmonary), although repair or replacement of the aortic or mitral valves is most common because they reside in the left side of the heart where pressures are the greatest.

A conventional heart valve replacement surgery involves accessing the heart in the patent's thoracic cavity through a longitudinal incision in the chest. For example, a median sternotomy requires cutting through the sternum and forcing the two opposing halves of the rib cage to be spread apart, allowing access to the thoracic cavity and heart within. The patient is then placed on cardiopulmonary bypass which involves stopping the heart to permit access to the internal chambers. After the heart has been arrested the aorta is cut open to allow access to the diseased valve for replacement. Such open heart surgery is particularly invasive and involves a lengthy and difficult recovery period.

Less invasive approaches to valve replacement have been proposed. The percutaneous implantation of a prosthetic valve is a preferred procedure because the operation is performed under local anesthesia, does not require cardiopulmonary bypass, and is less traumatic.

### BRIEF SUMMARY OF THE INVENTION

The present invention provides methods and devices for assessing the shape, size, topography, compliance, and other physical properties of a vessel lumen or a hollow portion of an organ located within a patient. The methods and devices may find use in the coronary vasculature, the atrial appendage, the peripheral vasculature, the abdominal vasculature, and in other ducts such as the biliary duct, the fallopian tubes, and similar lumen structures within the body of a patient. The methods and devices may also find use in the heart, lungs, kidneys, or other organs within the body of a patient. Moreover, although particularly adapted for use in vessels and organs found in the human body, the apparatus and methods may also find application in the treatment of animals.

However, the primary use of the methods and devices described herein is in the assessment of the size, shape, topography, compliance, spatial orientation, and other physical properties of the native heart valves. Such assessments are useful to facilitate proper orientation, sizing, selection, and implantation of prosthetic heart valves into the native valve space. Proper orientation, selection and sizing ensures that the prosthetic heart valve that is delivered during the implantation procedure will be of a size and shape that fits within the native valve space, including accommodations for any defects or deformities that are detected by the assessment process. Proper orientation, selection and sizing also ensures that the prosthetic valve, once fully expanded, will properly seal against the aortic wall to prevent leakage, and to prevent migration of the prosthetic valve.

The methods and devices described herein are suitable for use in facilitating the orientation, selection and sizing of prosthetic heart valves of all types, independent of the design, implantation mechanism, deployment technique, or any other aspect of the prosthetic valve. In many cases, particularly in the case of a prosthetic valve that is expandable from a delivery state to a deployed state, the assessment of the native valve space is of very great importance. For example, it is important to know the diameter of the native valve space when the valve space has been placed under the expansive load that is produced by the prosthetic valve. If the valve does not fit properly, it may migrate, leak, or resist deployment altogether.

The methods include use of an assessment member that is preferably located at or near the distal end of a catheter or other similar device. The assessment member is introduced to a treatment location within the patient, preferably the native cardiac valve, where the assessment member is activated or otherwise put into use to perform an assessment of one or more physical parameters of the treatment location, to collect the assessment information, and to provide the assessment information to the clinician. Assessment information includes the size (e.g., diameter, circumference, area, volume, etc.) of the valve space, the shape (e.g., round, spherical, irregular, etc.) of the lumen or hollow portion of the organ, the topography (e.g., locations, sizes, and shapes of any irregular features) of the lumen or hollow portion of the organ, the nature of any regular or irregular features (e.g., thrombosis, calcification, healthy tissue, fibrosa) and the spatial orientation (e.g., absolute location relative to a fixed reference point, or directional orientation) of a point or other portion of the treatment location. Access to the treatment location is obtained by any conventional method, such as by general surgical techniques, less invasive surgical techniques, or percutaneously. A preferred method of accessing the treatment location is transluminally, preferably by well-known techniques for accessing the vasculature from a location such as the femoral artery. The catheter is preferably adapted to engage and track over a guidewire that has been previously inserted and routed to the treatment site.

The assessment mechanism includes an expandable member that is attached to the catheter shaft at or near its distal end. The expandable member may comprise an inflatable balloon, a structure containing a plurality of interconnected metallic or polymeric springs or struts, an expandable “wisk”-like structure, or other suitable expandable member. In the case of an inflatable balloon, the expandable member is operatively connected to a source of inflation medium that is accessible at or near the proximal end of the catheter. The expandable member has at least two states, an unexpanded state and an expanded state. The unexpanded state generally corresponds with delivery of the assessment mechanism through the patient's vasculature. The expanded state generally corresponds with the assessment process. The expandable member is adapted to provide assessment information to the user when the expandable member is engaged with a treatment location within the body of a patient.

Turning to several exemplary devices and methods, in one aspect of the invention, a catheter-based system includes a transluminal imaging device contained partially or entirely within an expandable structure attached at or near the distal end of the catheter.

In a preferred embodiment, the imaging device is an ultrasonic imaging probe that is configured to transmit and receive ultrasonic signals at a desired frequency or at a plurality of desired frequencies. The expandable member has an acoustic reflective material. Sound waves emitted by the ultrasound imaging device are reflected differently by the acoustic reflective material than by the lumen or hollow portion of the organ. The received signals are then used to locate an outer periphery of the expandable member with respect to the shape and orientation of the lumen or hollow portion of the organ. In further preferred embodiments, the acoustic reflective material is on an inner wall of the expandable member, on an outer wall of the expandable member or embedded in the expandable member. The acoustic reflective material may be a material such as aluminum, gold, silver, or platinum. In addition, the acoustic reflective material may include microspheres made from silica, alumina, silver, gold, platinum or polymers. The microspheres may be sized on the order of nanometers and may be solid or hollow. The microspheres may be filled with air or another gas. In an exemplary embodiment, the microspheres are within a wall of the expandable member.

In the preferred embodiments, the expandable member is a balloon member. The balloon member is connected to an inflation lumen that runs between the proximal and distal ends of the catheter, and that is selectively attached to a source of inflation medium at or near the proximal end of the catheter. The balloon member is thereby selectively expandable while the imaging device is located either partially or entirely within the interior of the balloon. The imaging device is adapted to be advanced, retracted, and rotated within the balloon, thereby providing for imaging in a plurality of planes and providing the ability to produce three-dimensional images of the treatment site.

In optional embodiments, the expandable member is filled with a medium that enhances the imaging process. For example, the medium may comprise a material that increases the transmission capabilities of the ultrasonic waves, or that reduces the amount of scattering of the ultrasonic waves that would otherwise occur without use of the imaging-enhancing medium. In still other optional embodiments, the expandable structure contains (e.g., has embedded or formed within) or is formed of a material that enhances the imaging process. In still other embodiments, the expandable member includes a layer of or is coated with a material that enhances the imaging process.

In use, the transluminal imaging device is first introduced to the target location within the patient, such as the native valve annulus. In the preferred embodiment, this is achieved by introducing the catheter through the patient's vasculature to the target location. Typically, the catheter tracks over a guidewire that has been previously installed in any suitable manner. The imaging device may be provided with a radiopaque or other suitable marker at or near its distal end in order to facilitate delivery of the imaging device to the target location by fluoroscopic visualization or other suitable means. Once the imaging device is properly located at the target location, the expandable structure is expanded by introducing an expansion medium through the catheter lumen. The expandable structure expands such that it engages and applies pressure to the internal walls of the target location, such as the valve annulus. The expandable structure also takes on the shape of the internal surface of the target location, including all contours or other topography. Once the expandable structure has been sufficiently expanded, the imaging device is activated. Where appropriate, the imaging device is advanced, retracted, and/or rotated to provide sufficient movement to allow a suitable image of the target location to be created, or to collect a desired amount of measurement information. The measurement information collected and/or the images created by the imaging device are then transmitted to a suitable user interface, where they are displayed to the clinician.

In use, the expandable member is first introduced to the target location within the patient. In the preferred embodiment, this is achieved by introducing the catheter through the patient's vasculature to the target location. The catheter tracks over a guidewire that has been previously installed in any suitable manner. The expandable member carried on the catheter may be provided with a radiopaque or other suitable marker at or near its distal end in order to facilitate delivery of the physical assessment member to the target location by fluoroscopic visualization or other suitable means. Once the expandable member is properly located at the target location, the expandable member is expanded by introducing an expansion medium through the catheter lumen. The expandable member expands to a predetermined size such that the expandable member is able to engage the lumen or hollow portion of the organ, thereby providing an indicator of the shape and orientation of the lumen or hollow portion of the organ. In this way, the clinician is able to obtain precise measurements of the shape and orientation of the lumen or hollow portion of the organ at the target location. In a further preferred embodiment, the expandable member may be expanded to a size greater than the lumen or hollow portion of the organs to provide additional assessment information.

In a further aspect of the present invention, a valvuloplasty procedure is performed in association with the assessment of the native cardiac valve. In a first embodiment, the expandable member also functions as a valvuloplasty balloon. The expandable member is placed within the cardiac valve space, where it is expanded. Expansion of the expandable member causes the native valve to increase in size and forces the valve, which is typically in a diseased state in which it is stiff and decreased in diameter, to open more broadly. The valvuloplasty procedure may therefore be performed prior to the deployment of a prosthetic valve, but during a single interventional procedure. In a further preferred embodiment, the expandable member after performing valvuloplasty may be expanded beyond the shape and size of the native cardiac valve to distort the native cardiac valve and perform an assessment function.

The measurement and diagnostic processes performed by any of the foregoing devices and methods may be used to facilitate any suitable medical diagnosis, treatment, or other therapeutic processes. One particular treatment that is facilitated by the foregoing devices and methods is the repair and/or replacement of coronary valves, particularly aortic valve replacement using a prosthetic valve.

Other aspects, features, and functions of the inventions described herein will become apparent by reference to the drawings and the detailed description of the preferred embodiments set forth below.

### DESCRIPTION OF THE DRAWINGS

FIG. 1 is a perspective view of a catheter in accordance with several of the embodiments of the present invention.

FIG. 2A is a cross-sectional view of an imaging device in accordance with the present invention.

FIG. 2B is a cross-sectional view of the imaging device of FIG. 2A, showing an expandable member in its expanded state.

FIG. 3A is a cross-sectional view of an exemplary embodiment of an ultrasonic imaging device having an acoustic reflective material and FIGS. 3B-D are cross-sectional views showing various implementations of the acoustic reflective material.

FIG. 4 is a cross-sectional view of another exemplary embodiment of an imaging device having a reflective material.

FIGS. 5A to 5D are illustrations showing reflection of sound waves from an ultrasonic imaging device wherein FIG. 5A has no acoustic reflective material, FIG. 5B illustrates an acoustic reflective material on the inside of the expandable member, FIG. 5C illustrates an acoustic reflective material on the outside of the expandable member, and FIG. 5D illustrates an acoustic reflective material within a wall of the expandable member.

FIGS. 6A-C are cross-sectional views of the ultrasonic imaging device in various stages of expansion.

### DETAILED DESCRIPTION OF THE PREFERRED EMBODIMENTS

The present invention is directed to methods and devices for assessing the orientation, shape, size, topography, contours, and other aspects of anatomical vessels and organs using minimally invasive surgical techniques. As summarized above, the devices are typically catheter-based devices. Such devices are suitable for use during less invasive and minimally invasive surgical procedures. However, it should be understood that the devices and methods described herein are also suitable for use during surgical procedures that are more invasive than the preferred minimally invasive techniques described herein.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which these inventions belong. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise.

As will be apparent to those of skill in the art upon reading this disclosure, each of the individual embodiments described and illustrated herein has discrete components and features which may be readily separated from or combined with the features of any of the other several embodiments without departing from the scope or spirit of the present inventions.

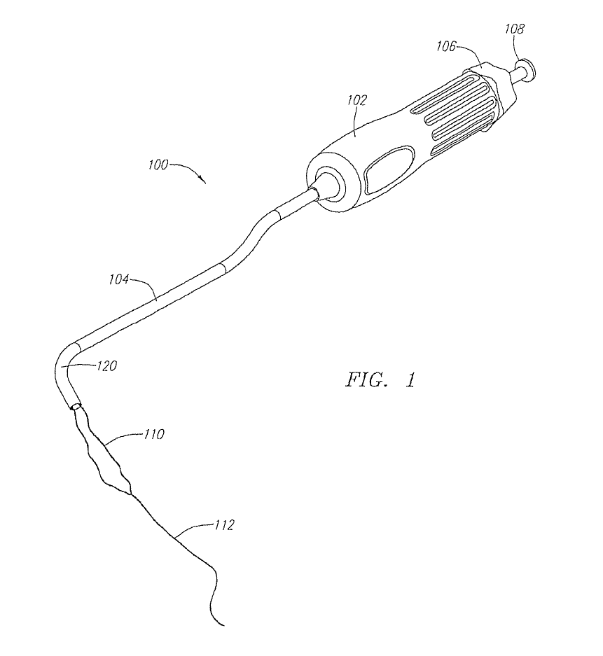

Turning to the drawings, FIG. 1 shows a catheter 100 suitable for use with each of the assessment mechanisms described herein. The catheter 100 includes a handle 102 attached to the proximal end of an elongated catheter shaft 104. The size and shape of the handle 102 may vary, as may the features and functionality provided by the handle 102. In the illustrated embodiment, the handle 102 includes a knob 106 rotatably attached to the proximal end of the handle 102. The knob 106 may be rotated to control the movement and/or function of one or more components associated with the catheter 100, such as for retraction of one or more catheter shafts or sheaths, or manipulation of an expandable member or other component carried at or near the distal end of the catheter shaft 104. Alternative structures may be substituted for the knob 106, such as one or more sliders, ratchet mechanisms, or other suitable control mechanisms known to those skilled in the art.

An inflation port 108 is located near the proximal end of the handle 102. The inflation port 108 is operatively connected to at least one inflation lumen that extends through the catheter shaft 104 to an expandable member 110 located near the distal end of the catheter shaft 104. The inflation port 108 is of any suitable type known to those skilled in the art for engaging an appropriate mechanism for providing an inflation medium to inflate the expandable member 110. For example, a suitable inflation mechanism is an Indeflator™ inflation device, manufactured by Guidant Corporation.

The catheter 100 is adapted to track a guidewire 112 that has been previously implanted into a patient and routed to an appropriate treatment location. A guidewire lumen extends through at least the distal portion of the catheter shaft 104, thereby providing the catheter 100 with the ability to track the guidewire 112 to the treatment location. The catheter 100 may be provided with an over-the-wire construction, in which case the guidewire lumen extends through the entire length of the device. Alternatively, the catheter 100 may be provided with a rapid-exchange feature, in which case the guidewire lumen exits the catheter shaft 104 through an exit port at a point nearer to the distal end of the catheter shaft 104 than the proximal end thereof.

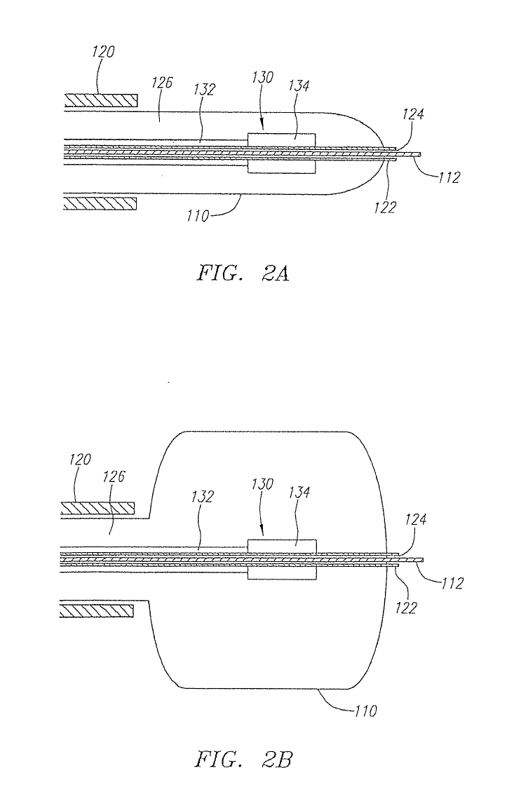

Turning next to FIGS. 2A-B, an assessment mechanism is shown and described. The assessment mechanism is located at the distal end of a catheter 100, such as that illustrated in FIG. 1 and described above. The assessment mechanism shown in FIGS. 2A-B includes an imaging device that is used to provide two-dimensional or three-dimensional images of a vessel lumen or the hollow portion of an organ within the body of a patient, as described below.

The assessment mechanism includes the outer sheath 120 of the catheter shaft 104, which surrounds the expandable member 110. In the preferred embodiment, the expandable member 110 is an inflatable balloon. The expandable member 110 is attached at its distal end to a guidewire shaft 122, which defines a guidewire lumen 124 therethrough. The guidewire 112 extends through the guidewire lumen 124.

An imaging member 130 is contained within the expandable member 110. The imaging member 130 is supported by a shaft 132 that extends proximally to the handle 102, where it is independently controlled by the user. The imaging member shaft 132 is coaxial with and surrounds the guidewire shaft 124, but is preferably movable (e.g., by sliding) independently of the guidewire shaft 124. At the distal end of the imaging member shaft 132 is the imaging head 134. The imaging head 134 may be any mechanism suitable for transmitting and receiving ultrasonic waves. In a preferred embodiment, there may be a plurality of imaging heads 134, although only one such imaging head 134 is shown for clarity. A typical imaging head 134 is an ultrasonic imaging probe. The expandable member 110 is subject to expansion when a suitable expansion medium is injected into the expandable member through the inflation lumen 126. The inflation lumen 126, in turn, is connected to the inflation port 108 associated with the handle 102. FIG. 2A illustrates the expandable member 110 in its unexpanded (contracted) state, while FIG. 2B illustrates the expandable member 110 in its expanded state, such as after a suitable inflation medium is injected through the inflation port 108 and inflation lumen 126 into the expandable member 110.

To use the assessment mechanism illustrated in FIGS. 2A-B, the distal portion of the catheter is delivered to a treatment location within the body of a patient over the previously deployed guidewire 112. In a particularly preferred embodiment, the treatment location is the aortic heart valve, and the guidewire 112 is deployed through the patient's vasculature from an entry point in the femoral artery using, for example, the Seldinger technique. Deployment of the assessment mechanism is preferably monitored using fluoroscopy or other suitable visualization mechanism. Upon encountering the treatment location, the expandable member 110 is expanded by inflating the balloon with a suitable inflation medium through the inflation port 108 and the inflation lumen 126. The expandable member 110 engages the internal surfaces of the treatment location, such as the annular root of the aortic heart valve. Once the expandable member 110 is expanded, the imaging head 134 is activated and the imaging process is initiated. The imaging head 134 is preferably advanced, retracted, and rotated within the expandable member 110 as needed to obtain images in a variety of planes to yield a 360° three-dimensional image, or any desired portion thereof. Once the imaging process is completed, the expandable member 110 is deflated, and the assessment mechanism may be retracted within the catheter shaft 104. The catheter 100 is then removed from the patient.

Optionally, the inflation medium used to expand the expandable member 110 may comprise a material that enhances the ability of the imaging head 134 to generate images. For example, the inflation medium may facilitate enhanced acoustic transmission, reception, or it may reduce the incidence of scattering of the assessment signal. Such suitable inflation media may include a liquid or a gas and more specifically may include, for example, the following: acoustic gel, dielectric fluid, saline, blood, gas, contrast medium and the like. These effects may be enhanced further by provision of a material or coating on the surface of the expandable member 110 that optimizes the imaging process. Such suitable materials and/or coatings include relatively dense materials such as metal, ceramic, high density polymers, and the like.

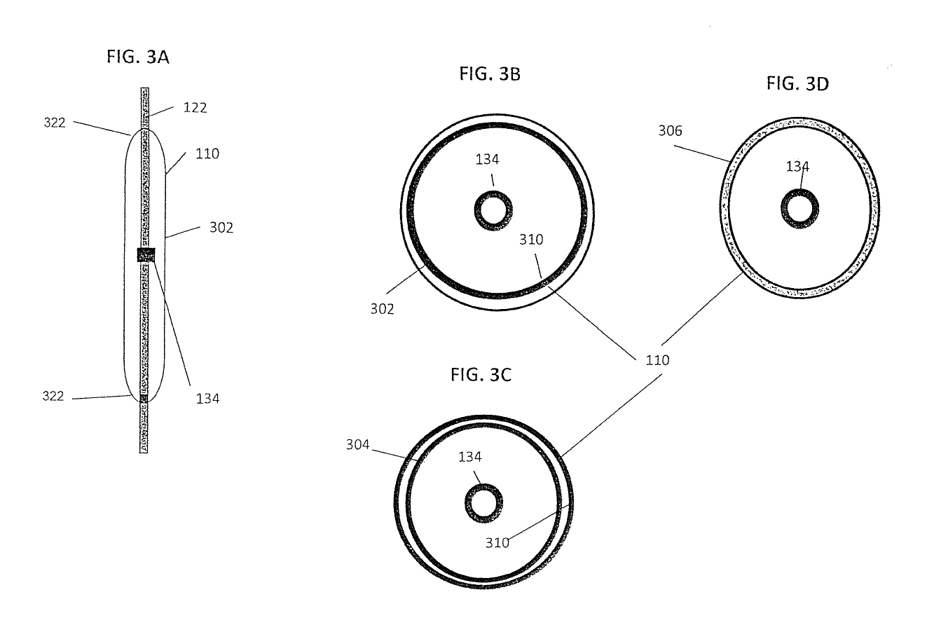

A preferred embodiment is shown in FIGS. 3A-D. In FIG. 3A, expandable member 110 is shown with guidewire shaft 122. Within expandable member 110 is located imaging head 134 which is an ultrasound imaging device such as a piezoelectric transducer. The expandable member 110 also has an acoustic reflective material which may be coated on the inside or outside of the expandable member 110 or may be incorporated within the material of the expandable member 110. In a preferred embodiment, the acoustic reflective material extends the entire length, or nearly the entire length, of the expandable member 110 so as to provide enhanced acoustic reflection over substantially the entire length of the expandable member 110. There need not be acoustic reflective material near the ends 322 of the expandable members 110 where the expandable member 110 is joined to the catheter. A cross section of the expandable member 110, imaging head 134 and acoustic reflective material 302 coated on the outside surface of the expandable member 110 is shown in more detail in FIG. 3B. The acoustic reflective material may be any material that substantially reflects sound waves such as aluminum, gold, silver, or platinum. In addition, the acoustic reflective material may include microspheres made from silica, alumina, silver, gold, platinum, or polymers. The microspheres may be sized on the order of nanometers and may be solid or hollow. The microspheres may be filled with air or another gas. In an exemplary embodiment, the microspheres are within a wall of the expandable member. In other exemplary embodiments, the acoustic reflective material may be present as a coating 304 on the inner wall 310 of the expandable member 110 as shown in FIG. 3C or embedded 306 within the material of the expandable member 110 as shown in FIG. 3D.

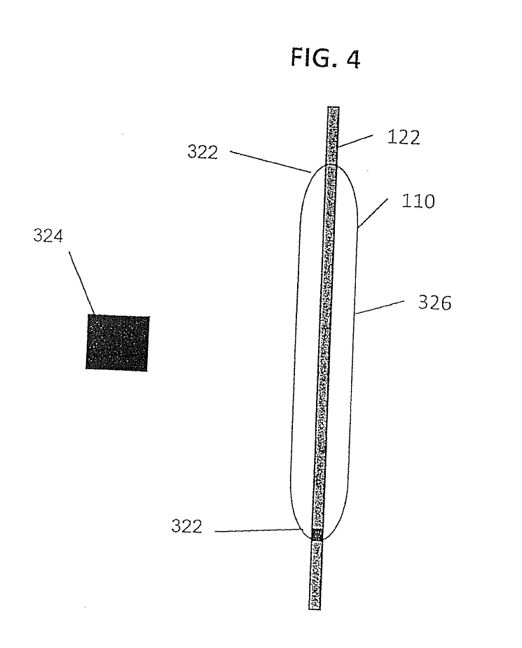

A further embodiment of the present invention is shown in FIG. 4. FIG. 4 is similar to FIG. 3A except that there is no imaging head contained within the expandable member 110. In addition, while the expandable member 110 does contain a reflective material 326 it need not be an acoustic reflective material. The reflective material 326 may be on or within a wall of the expandable member 110 similarly to that shown in FIGS. 3B, 3C and 3D. The embodiment shown in FIG. 4 may be used with any means for imaging, including but not limited to, ultrasound imaging, optical coherence tomography (OCT), or acoustic imaging by transesophageal echo. If ultrasound imaging is utilized, then the reflective material is an acoustic reflective material while if OCT is utilized, the reflective material is an optical reflective material. Means of imaging may be used other than those means of imaging disclosed herein. Moreover, the imaging device may be located outside of the expandable member 110. For example, an ultrasound imaging device 324 may be located external to the lumen such as outside the body of the patient as schematically shown in FIG. 4.

Acoustic impedance may be calculated according to the following equation:

Z=ρ·c

where
Z is the characteristic acoustic impedance of a material;
ρ is the density of the medium; and
c is the longitudinal sound speed.

Referring now to FIG. 3C, an example of the reflection due to the acoustic reflective material is illustrated using the above principles of acoustic impedance and reflection. An imaging head 134 is contained within an expandable member 110. On the inside wall 310 of the expandable member 110 is a coating 304 of acoustic reflective material. The expandable member is shown as being inserted within a vessel lumen. Included within expandable member 110 is a medium for expanding the expandable member such as a saline fluid.

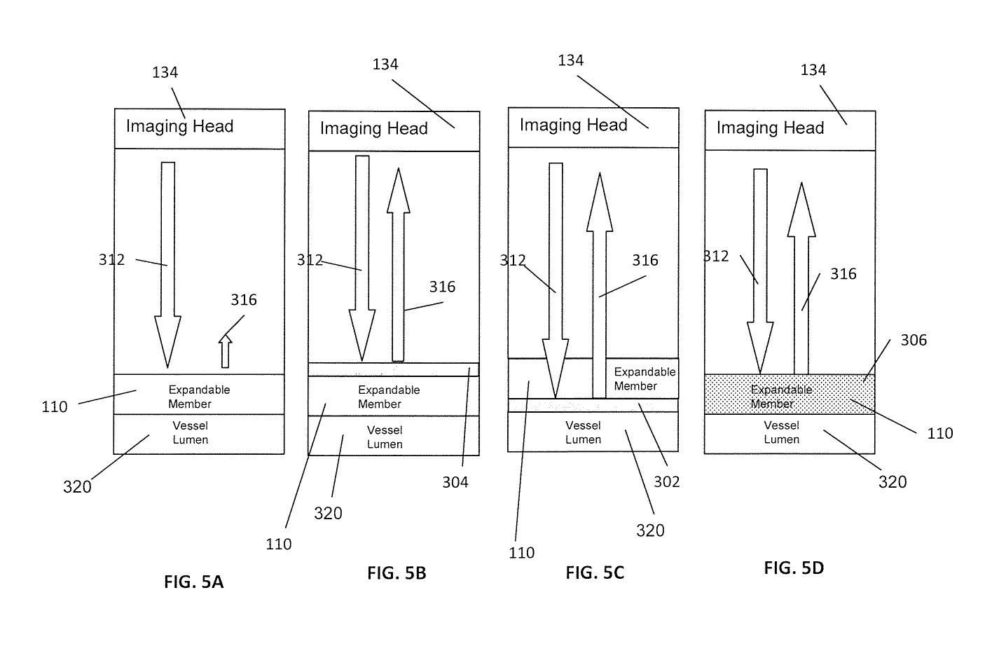

When the imaging head 134 emits sound waves, the sound waves will easily pass through the expandable member 110 and into the vessel lumen 320. Referring to FIG. 5A, it can be seen that little of the sound waves are reflected back to the imaging head 134 as indicated by the different size of the arrows where arrow 312 represents the emitted sound waves while arrow 316 represents the reflected sound waves. In FIG. 5B, there is an acoustic reflective material 304 on the inside of the expandable member which causes a much greater proportion of sound waves to be reflected back (represented by arrow 316) to the imaging head 134. Similarly, in FIG. 5C where an acoustic reflective material 302 is on an outside of the expandable member 110 and FIG. 5D where an acoustic reflective material 306 is embedded within a wall of the expandable member 110, the reflected sound waves represented by arrow 316 are very close in proportion to the emitted sound waves represented by arrow 312. FIGS. 5B-5D illustrate that a large proportion of the emitted sound waves are reflected back to the imaging head 134 due to the presence of the acoustic reflective materials 302, 304, 306. A higher proportion of the emitted sound waves reflected back to the imaging head 134 by the acoustic reflective materials 302, 304, 306 will improve the locating of the edge of the expandable member 110 with respect to the vessel lumen and, more particularly, with respect to a cardiac valve. Such locating will allow a clinician to determine the shape and orientation of the cardiac valve.

In general, it is preferred for the acoustic reflective material to have an acoustic impedance Z₁ different than the acoustic impedance Z₃ of expandable member 110 and the acoustic impedance Z₂ of the medium (such as the saline) for expanding the expandable member 110. More specifically, in one exemplary embodiment, the acoustic reflective material may have an acoustic impedance Z₁ greater than the acoustic impedance Z₃ of expandable member 110 and the acoustic impedance Z₂ of the expanding medium. In another exemplary embodiment, the acoustic reflective material may have an acoustic impedance Z₁ less than the acoustic impedance Z₃ of expandable member 110 and acoustic impedance Z₂ of the expanding medium. In either of the foregoing exemplary embodiments, the reflection of the expandable member 110 is enhanced so as to differentiate itself from the vessel lumen, hollow organ and expanding medium.

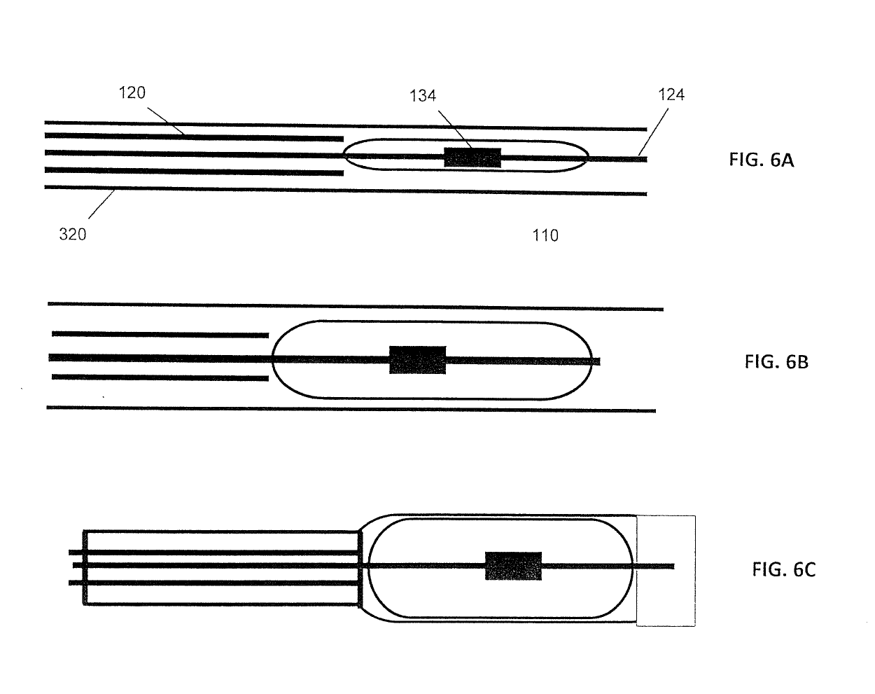

Referring now to FIGS. 6A-C, a further exemplary embodiment is illustrated. FIG. 6A shows the expandable member 110 within a vessel lumen 320. The expandable member 110 is in the unexpanded state. Also shown in FIGS. 6A-C are the outer sheath 120 of the catheter shaft, guidewire lumen 124 and imaging head 134. In FIG. 6B, the expandable member 110 has been expanded to be in contact with the vessel lumen 320. Vessel lumen may also be a native cardiac valve. At this point, expandable member 110 may be called upon to do a procedure such as valvuloplasty, for example. Thereafter, expandable member 110 may be expanded further as shown in FIG. 6C to perform a measurement and/or diagnostic assessment such as any of those described previously. The expansions of expandable member 110 shown in FIGS. 6B and 6C may be done serially without first withdrawing the expandable member 110 between expansions.

The preferred embodiments of the inventions that are the subject of this application are described above in detail for the purpose of setting forth a complete disclosure and for the sake of explanation and clarity. Those skilled in the art will envision other modifications within the scope and spirit of the present disclosure. Such alternatives, additions, modifications, and improvements may be made without departing from the scope of the present inventions, which is defined by the claims.

What is claimed is:

1. A device for assessing the shape and orientation of a lumen or hollow portion of an organ comprising:
a catheter shaft having a proximal end and a distal end;
an expandable member carried at or near the distal end of the catheter, the expandable member having an acoustic reflective material; and
an ultrasound imaging head contained within the expandable member, the ultrasound imaging head capable of emitting and receiving sound waves;
wherein, in operation, the sound waves emitted by the ultrasound imaging head are reflected by the acoustic reflective material so as locate the expandable member with respect to the lumen or hollow portion of the organ.

2. The device of claim 1 wherein the acoustic reflective material is on an inner surface of a wall of the expandable member.

3. The device of claim 1 wherein the acoustic reflective material is on an outer surface of a wall of the expandable member.

4. The device of claim 1 wherein the acoustic reflective material is within a wall of the expandable member.

5. The device of claim 1 wherein the acoustic reflective material is along substantially an entire length of the expandable member.

6. The device of claim 1 wherein a wall of the expandable member is acoustically reflective.

7. The device of claim 1 wherein there are a plurality of ultrasound imaging heads.

8. The device of claim 1 wherein the ultrasound imaging head is external to the expandable member.

9. The device of claim 1 wherein the lumen is a cardiac valve, atrial appendage, coronary lumen, peripheral lumen, abdominal lumen, biliary duct or fallopian tube.

10. The device of claim 1 wherein the sound waves emitted by the ultrasound imaging head are reflected by the acoustic reflective material so as to locate the expandable member with respect to the shape and orientation of the lumen.

11. The device of claim 1 wherein the expandable member is capable of performing a valvuloplasty procedure.

12. The device of claim 1 further comprising a medium for expanding the expandable member.

13. The device of claim 12 wherein the medium is selected from the group consisting of saline, acoustic gel, dielectric fluid, blood, gas and contrast medium.

14. The device of claim 12 wherein the medium is a liquid or a gas.

15. The device of claim 12 wherein the acoustic reflective material has an acoustic impedance Z₁, the medium has an acoustic impedance Z₂ and the expandable member has an acoustic impedance Z₃ such that Z₁ is different than Z₂ and Z₃.

16. The device of claim 12 wherein the acoustic reflective material has an acoustic impedance Z₁, the medium has an acoustic impedance Z₂ and the expandable member has an acoustic impedance Z₃ such that Z₁ is greater than Z₂ and Z₃.

17. The device of claim 12 wherein the acoustic reflective material has an acoustic impedance Z₁, the medium has an acoustic impedance Z₂ and the expandable member has an acoustic impedance Z₃ such that Z₁ is less than Z₂ and Z₃.

18. The device of claim 1 wherein the expandable member is a balloon.

19. A device comprising:
a catheter shaft having a proximal end and a distal end;
an expandable member carried at or near the distal end of the catheter, the expandable member having an acoustic reflective material on or within the expandable member.

20. The device of claim 19 wherein the acoustic reflective material extends substantially an entire length of the expandable member.

21. The device of claim 19 wherein the acoustic reflective material is on an inner surface of a wall of the expandable member.

22. The device of claim 19 wherein the acoustic reflective material is on an outer surface of a wall of the expandable member.

23. The device of claim 19 wherein the acoustic reflective material is within a wall of the expandable member.

24. The device of claim 19 wherein the expandable member is a balloon.

25. A device comprising:
a catheter shaft having a proximal end and a distal end;
an expandable member carried at or near the distal end of the catheter, the expandable member having an acoustic reflective material on or within the expandable member, the acoustic reflective material enhancing the reflectivity of the expandable member.

26. The device of claim 25 wherein the acoustic reflective material reduces the reflectivity of the expandable member.

27. The device of claim 25 wherein the acoustic reflective material increases the reflectivity of the expandable member.

28. A device comprising:
a catheter shaft having a proximal end and a distal end;
an expandable member carried at or near the distal end of the catheter, the expandable member having a reflective material on or within the expandable member.

29. The device of claim 28 wherein the reflective material is an acoustically reflective material.

30. The device of claim 28 wherein the reflective material is an optically reflective material.

31. A method for assessing a lumen or hollow portion of an organ comprising:
deploying an expandable member having an acoustic reflective material and an ultrasound imaging head to the location of a lumen or hollow portion of an organ within the body of a patient, the expandable member being attached to a catheter at or near a distal end thereof;
expanding the expandable member to engage at least a portion of the lumen or hollow portion of an organ;
emitting sound waves by the ultrasound imaging head;
receiving sound waves reflected by the acoustic reflective material so as to locate the expandable member with respect to the lumen or hollow portion of an organ.

32. The method of claim 31 wherein the acoustic reflective material is on an inner surface of a wall of the expandable member.

33. The method of claim 31 wherein the acoustic reflective material is on outer inner surface of a wall of the expandable member.

34. The method of claim 31 wherein the acoustic reflective material is within a wall of the expandable member.

35. The method of claim 31 wherein a wall of the expandable member is acoustically reflective.

36. The method of claim 31 wherein there are a plurality of ultrasound imaging heads and emitting sound waves includes emitting sound waves by the plurality of ultrasound imaging heads.

37. The method of claim 31 wherein the ultrasound imaging head is external to the expandable member.

38. The method of claim 31 wherein the lumen is a cardiac valve, atrial appendage, coronary lumen, peripheral lumen, abdominal lumen, biliary duct or fallopian tube.

39. The method of claim 31 wherein receiving sound waves reflected by the acoustic reflective material so as to locate the expandable member with respect to the shape and orientation of the lumen.

40. The method of claim 31 further comprising a medium for expanding the expandable member.

41. The method of claim 40 wherein the medium is selected from the group consisting of saline, acoustic gel, dielectric fluid, blood, gas and contrast medium.

42. The method of claim 40 wherein the medium is a liquid or a gas.

43. The method of claim 40 wherein the acoustic reflective material has an acoustic impedance Z₁, the medium has an acoustic impedance Z₂ and the expandable member has an acoustic impedance Z₃ such that Z₁ is different than Z₂ and Z₃.

44. The method of claim 40 wherein the acoustic reflective material has an acoustic impedance Z₁, the medium has an acoustic impedance Z₂ and the expandable member has an acoustic impedance Z₃ such that Z₁ is greater than Z₂ and Z₃.

45. The method of claim 40 wherein the acoustic reflective material has an acoustic impedance Z₁, the medium has an acoustic impedance Z₂ and the expandable member has an acoustic impedance Z₃ such that Z₁ is less than Z₂ and Z₃.

46. The method of claim 31 wherein the expandable member is a balloon.

47. The method of claim 31 wherein after the step of expanding, further comprising further expanding the expandable member so as to expand the lumen or hollow portion of the organ valve outwardly.

48. The method of claim 47 wherein during the step of further expanding, further comprising performing a valvuloplasty procedure.

49. The method of claim 47 wherein during the step of further expanding, further comprising performing an assessment of the lumen or hollow portion of the organ.

50. The method of claim 47 wherein the steps of expanding and further expanding are done serially without withdrawing the expandable member from the lumen or hollow portion of the organ.
